(19) United States
(12) Patent Application Publication
(10) Pub. No.: US 2026/0294372 A1
(43) Pub. Date: Oct. 1, 2026

(54) AUXILIARY ASSESS METHOD AND SYSTEM TRAINED ON A SMALL AMOUNT OF CHEST X-RAY IMAGES

(71) Applicant: National Defense Medical University, Taipei City (TW)

(72) Inventors: Wen-Hui Fang, Taipei (TW); Chin Lin, Taipei (TW); Kai-Chieh Chen, Taipei (TW); Wei-Chou Chang, Taipei (TW); Yi-Chih Hsu, Taipei (TW); Kai-Hsiung Ko, Taipei (TW); Shing-An Lin, Taipei (TW)

(21) Appl. No.: 19/089,020

(22) Filed: Mar. 25, 2025

(51) Int. Cl. A61B 6/50; A61B 6/00; G06V 10/44; G16H 30/20; G16H 30/40

(52) U.S. Cl. CPC A61B 6/50; A61B 6/5211; G06V 10/44; G16H 30/20; G16H 30/40

(57) ABSTRACT

An auxiliary assess method trained on a small amount of chest X-ray images includes the steps of acquiring and storing chest X-ray images each of which includes a certified text report and a structured token of at least one target disease, preprocessing the chest X-ray images to turn the chest X-ray images into preprocessed chest X-ray images in a same specification, building a contrastive learning model of at least one target disease from the preprocessed chest X-ray images, using the contrastive learning model to extract high-level features from the preprocessed chest X-ray images, conducting linear detection on the extracted high-level features of the processed chest X-ray images, using the contrastive learning model to build a target prediction model of at least one target disease, and providing assessed chest X-ray images with tokens of corresponding target diseases marked by the target prediction model.

### BACKGROUND OF INVENTION

### 1. Field of Invention

The present invention relates to assess for helping with diagnosis and, more particularly, to an auxiliary assess method and system trained on a small amount of chest X-ray images to provide accuracy close to that of calculation executed by a deep convolutional neural network trained on full samples.

### 2. Related Prior Art

Chest X-ray (“CXR”) imaging is an imaging technique that is used at least 2 billion times a year worldwide. In the development of CXR analysis techniques, deep learning techniques, especially convolutional neural networks (“CNNs”), are found to achieve expert-level conductance in supervised learning models. However, the success of supervised learning models depends heavily on a large amount of data and high-quality annotations. Sometimes, it requires experts to collaborate to generate high-quality labels. This practice consumes a lot of time, manpower and cost. The lack of large and high-quality annotated datasets gravely affects supervised deep learning of medical imaging tasks. Therefore, it is a critical technical challenge to develop appropriate methods to reduce the dependence of supervised learning models on large structured annotated datasets.

Therefore, the present invention is intended to obviate or at least alleviate the problems encountered in the prior art.

### SUMMARY OF INVENTION

It is the primary objective of the present invention to provide an auxiliary assess method trained on a small amount of chest X-ray images.

To achieve the foregoing objective, the auxiliary assess method includes the steps of acquiring and storing chest X-ray images each of which includes a certified text report and a structured token of at least one target disease, preprocessing the chest X-ray images to turn the chest X-ray images into preprocessed chest X-ray images in a same specification, building a contrastive learning model of at least one target disease from the preprocessed chest X-ray images, using the contrastive learning model to extract high-level features from the preprocessed chest X-ray images, conducting linear detection on the extracted high-level features of the processed chest X-ray images, using the contrastive learning model to build a target prediction model of at least one target disease, and providing assessed chest X-ray images with tokens of corresponding target diseases marked by the target prediction model.

Other objectives, advantages and features of the present invention will be apparent from the following description referring to the attached drawings.

### BRIEF DESCRIPTION OF DRAWINGS

The present invention will be described via detailed illustration of the preferred embodiment referring to the drawings wherein:

FIG. 1 is a flow chart of an auxiliary assess method trained on a small amount of chest X-ray images according to the preferred embodiment of the present invention; and

FIG. 2 is a block diagram of a system for executing the auxiliary assess method shown in FIG. 1.

### DETAILED DESCRIPTION OF PREFERRED EMBODIMENT

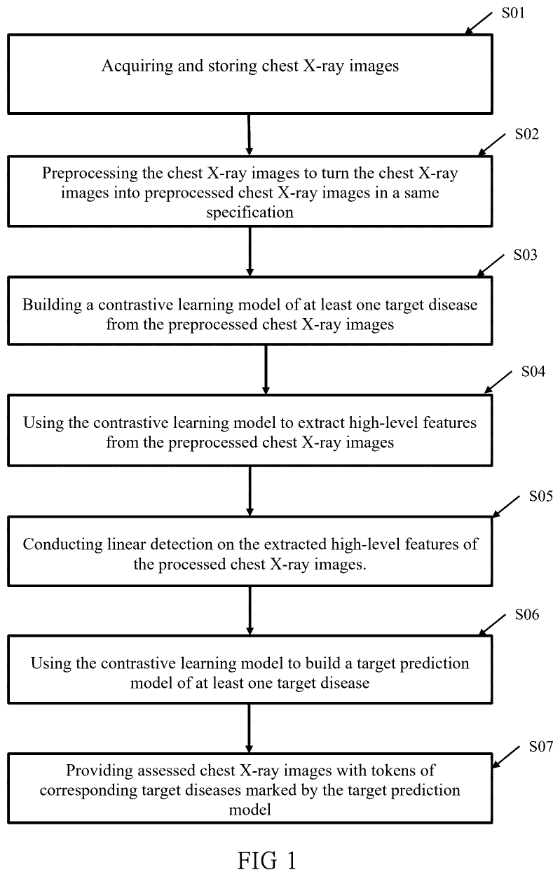

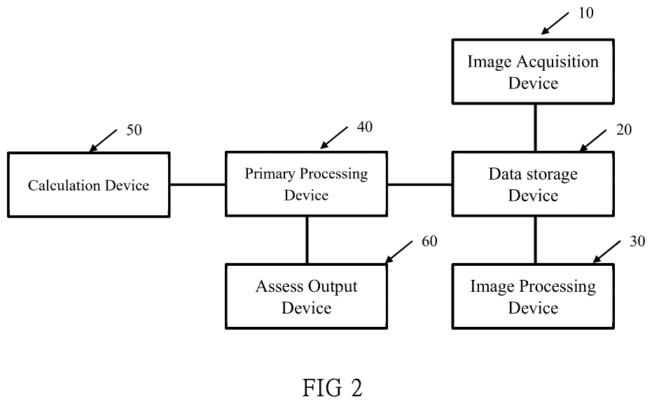

Referring to FIGS. 1 and 2, according to the preferred embodiment of the present invention, an auxiliary assess method trained on a small amount of chest X-ray images is executed in an auxiliary assess system.

Referring to FIG. 2, the auxiliary assess system includes an image acquisition device 10, a data storage device 20, an image processing device 30, a primary processing device 40, a calculation device 50 and a assess output device 60. The image acquisition device 10 is electrically connected to the data storage device 20. The image processing device 30 is also electrically connected to the data storage device 20. The data storage device 20 is electrically connected to the primary processing device 40. The calculation device 50 is also electrically connected to the primary processing device 40. The primary processing device 40 is electrically connected to the assess output device 60.

The image acquisition device 10, the data storage device 20, the image processing device 30, the primary processing device 40, the calculation device 50 and the assess output device 60 can be made in one piece or individual devices. Where the image acquisition device 10, the data storage device 20, the image processing device 30, the primary processing device 40, the calculation device 50 and the assess output device 60 are individual devices, they are in communication of data with one another in a wireless manner or via wires.

Referring to FIG. 1, the auxiliary assess method trained on a small amount of chest X-ray images is operable to use a deep convolutional neural network to calculate chest X-ray images to quickly predict the probabilities of diseases related to the chest. Preferably, the auxiliary assess method includes S01 through S07 as follows.

At S01, the image acquisition device 10 acquires chest X-ray images. The image acquisition device 10 acquires anteroposterior position (AP) or posteroanterior (PA) chest X-ray images and transfers the chest X-ray images into the data storage device 20 in which the chest X-ray images are stored. Each chest X-ray image contains a text report certified by a qualified physician and a structured label of at least one target disease marked by a certified radiologist. It is ensured that only one chest X-ray image is selected for each patient to be stored in the data storage device 20 to reduce potential problems associated with sample interdependence in statistical analysis.

Preferably, the image acquisition device 10 is an X-ray scanner operable to provide fresh chest X-ray images or a device operable to acquire existent chest X-ray images from a database of a medical institution, atelectasis, atherosclerosis, cardiomegaly, consolidation change, costophrenic angle blunting, degenerative joint disease, emphysematous change, endotracheal tubes, fracture, inflammatory, malignancy, nasogastric tubes, osteoarthritis, osteoporosis, osteophyte formation, pacemaker, perm catheter insertion, pigtail or drainage, pleural effusion, pneumonia, pneumothorax, Port A implantation, prominence of hilar shadow, pulmonary edema, scalloping of the diaphragm, spondylosis, sternotomy, tracheostomy, vertebroplasty and widening of the mediastinum. The target diseases can further include, but are not limited to, five diseases related to chest X-ray images such as left ventricular dysfunction, aortic stenosis, pulmonary arterial hypertension, left atrial enlargement and pericardial effusion. These chest X-ray images are annotated with structural reports of echocardiographs of the diseases.

At S02, the image processing device 30 preprocesses the chest X-ray images. The image processing device 30 adjusts the specifications of the chest X-ray images so that the processed chest X-ray images are in a same specification. For example, the pixel sizes of the chest X-ray images stored in the data storage device 20 may not be identical to one another. The image processing device 30 adjusts the pixel sizes of the chest X-ray images stored in the data storage device 20 while maintaining the aspect ratio of each chest X-ray image, thereby providing preprocessed chest X-ray images in a same specification. For example, the image processing device 30 sets the short side of each chest X-ray image to be 256 pixels for scaling, normalizes the numerical range of each pixel to be 8 digits, sets the length of the text report for each chest X-ray image to be no more than 256 tokens such as the GPT-3.5-TURBO-0613 model, and provides a prompt such as “Please summarize and condense the following report: the original radiology report” until the text report of each preprocessed chest X-ray image includes no more than 256 tokens. Then, the image processing device 30 transfers the preprocessed chest X-ray images to the data storage device 20 in which the preprocessed chest X-ray images are stored.

At S03, the primary processing device 40 builds a contrastive learning model of at least one of the target diseases from the preprocessed chest X-ray image. In specific, the primary processing device 40 obtains the preprocessed chest X-ray images from the data storage device 20 and then uses contrastive language-image pretraining (“CLIP”) to establish a contrastive learning model.

The primary processing device 40 includes an image encoder and a text encoder (not shown). For example, the image encoder uses ViT-B/32 to divide each preprocessed chest X-ray image that includes 256×256 pixels into an 8×8 matrix via a 32×32 convolutional neural network. Then, each 8 X8 matrix is adjusted to be a 64×1 matrix. Then, a token for classification (“CLS”) is added to each 64×1 matrix as the first digit, thereby turning each 64×1 matrix into a 65×1 matrix. Then each 65×1 matrix is transferred into a transformer that is 12-layer deep and includes a hidden layer of 768 in size. Eventually provided is a vector that is 512 in size and includes a CLS token.

Preferably, the text encoder is a standard language encoder that first tokenizes the text into vectors with a maximum length of 256, and then adds a CLS token to each vector as the first token. Then, the standard language encoder, which is 12-layer deep and includes an embedding layer in a size of 512, a hidden layer in a size of 768 and 8 attention heads eventually provides a 512-sized vector with the CLS token.

As discussed above, each preprocessed chest X-ray image or text report is added with the corresponding CLS token before it is fed into the corresponding encoder. The CLS tokens are embeddings for the chest X-ray images and text reports. Finally, in the backpropagation process, the CLS tokens of the chest X-ray images and the CLS tokens of the text reports are calculated to obtain a similarity matrix which is then optimized into the final output via softmax and cross-entropy loss.

Preferably, all technical details strictly follow OpenAI's CLIP model, and the weights of the model are used as initialization parameters. In the pretraining, the training set is randomly divided into 90% for model fitting and 10% for validation. All parameters were fine-tuned by the standard parameters of an SGD optimizer, with a batch size of 64, a learning rate of 0.0001 and a momentum of 0.9. Throughout the training, each chest X-ray image is randomly cropped to 256×256 pixels. The model is trained for 50 cycles, and the validation loss is calculated at the end of each cycle. Thus, the best-conducting model can be selected. Preferably, the model training is carried out in Python environment version 3.10.10, using the “torch” package version 2.0.1.

At S04, the primary processing device 40 uses the contrastive learning model to extract the high-level features of the preprocessed chest X-ray images. In specific, the primary processing device 40 uses the trained contrastive learning model to extract the high-level features from the preprocessed chest X-ray images stored in the data storage device 20. Each high-level feature is a vector of 512 in size. A small-scale sample search is carried out for selecting the samples. Preferably, the ratio of the control group to the case group (with the target disease) is 3. For example, 128 samples are used in the case group, and 384 samples are used in the control group. According to the ratio, starting from 1, secondary samples are randomly selected. Then, secondary samples are oversampled.

At S05, the calculation device 50 conducted linear detection on the preprocessed chest X-ray images from which the high-level features have been extracted. In specific, after the extraction of the high-level features from the processed chest X-ray images in the pretrained model, the computing device 50 conducts the linear detection on the preprocessed chest X-ray images. Preferably, the “glmnet” package version 2.0-16 in R software 3.4.4 is used to conduct the linear detection on the CXR embeddings. The “glmnet” package uses an elastic network algorithm that includes L1 and L2 regularization terms in logistic regression. For each target disease, the samples are first oversampled to ensure that the number of the case samples is identical to the number of the control samples. Then, the “cv.glmnet” function is used to divide the samples into four subsets for cross-validation. In the cross-validation, the optimal value of lambda (regularized hyperparameter) is sought in the range of e-9 to e-1 (a logarithmically spaced sequence with a length of 45), and alpha (the ratio of L1 and L2 penalties) is sought in the range of 0 to 1 (with increments of 0.1) to maximize the cross-validation area under curve (“AUC”). The selected model fitting by the hyperparameter with the largest cross-validation AUC is subsequently used on the test set.

Preferably, the pretrained model using 128 case samples and 384 control samples for linear detection achieves accuracy close to that of a convolutional neural network trained on full samples.

At S06, the primary processing device 40 uses the pretrained model to build a target prediction model for at least one target disease. The target diseases include but are not limited to the above-mentioned 31 chest-related diseases and the above-mentioned 5 diseases associated with echocardiography.

At S07, the assess output device 60 provides a chest X-ray image corresponding to one of the key tokens of the target disease marked by the target prediction model. In specific, the image acquisition device 10 acquires a chest X-ray image of a patient to be tested, preprocess it, and adjusts it to the same specification as the pretrained preprocessed chest X-ray images stored in the data storage device 20 while maintaining the aspect ratio and setting the short side to 256 pixels for scaling. The primary processing device 40 conducts classification via the comparative learning model and the target prediction model. Thus, the chest X-ray image to be tested obtains the token of the corresponding target disease. The assess output device 60 provides an assess chest X-ray image pliant to these results. Different colors can be sued to show the tokens of the target diseases. The assessed chest X-ray image helps a medical personnel or interpreter understand the patient's physical condition and provides suggestions for follow-up treatment or surgery. The assess output device 60 can be any display such as a monitor of a desktop or laptop computer and a display of a handheld device.

Referring to FIG. 2, the system of the present invention is as revealed in the second figure, wherein the image acquisition device 10 is connected to the data storage device 20 for obtaining chest X-ray images and stored in the data storage device 20, wherein the chest X-ray images are accompanied by certified text reports and structured labels of at least one target disease, and the data storage device 20 is connected to the image processing device 30, the image processing device 30 can obtain such chest X-ray images from the data storage device 20 for preprocessing to form a corresponding preprocessed chest X-ray image of the same specification, the primary processing device 40 is also connected to the data storage device 20, the primary processing device 40 can establish a comparative learning model of the preprocessed chest X-ray image, and the processing device 40 is further connected to the computing device 50 to establish a target prediction model after extracting high-level features and linear detection through the operation of the computing device 50, so that after the chest X-ray image to be measured is operated by the primary processing device 40 and the target prediction model of the computing device 50, the marker of the corresponding target disease can be obtained in the chest X-ray image to be tested, and the primary processing device 40 is connected with the assess output device 60, and these results are output into a relative assess chest X-ray image through the assess output device 60 to assist medical personnel in judging the patient's status for real-time monitoring and intervention by medical personnel as recommendations for follow-up treatment or surgery.

The present invention has been described via the illustration of the preferred embodiment. Those skilled in the art can derive variations from the preferred embodiment without departing from the scope of the present invention. Therefore, the preferred embodiment shall not limit the scope of the present invention defined in the claims.

1. An auxiliary assess method trained on a small amount of chest X-ray images comprising the steps of:
acquiring chest X-ray images each of which includes a certified text report and a structured token of at least one target disease;
storing the plural chest X-rays;
preprocessing the chest X-ray images to turn the chest X-ray images into preprocessed chest X-ray images in a same specification;
building a contrastive learning model of at least one target disease from the preprocessed chest X-ray images;
using the contrastive learning model to extract high-level features from the preprocessed chest X-ray images;
conducting linear detection on the extracted high-level features of the processed chest X-ray images;
using the contrastive learning model to build a target prediction model of at least one target disease; and
providing assessed chest X-ray images with tokens of corresponding target diseases marked by the target prediction model.

2. The auxiliary assess method according to claim 1, wherein each of the chest X-ray images is one of an anteroposterior chest X-ray image and a posteroanterior chest X-ray image.

3. The auxiliary assess method according to claim 1, wherein the target disease is selected from the group of aneurysm, pulmonary insufficiency, atherosclerosis, cardiac hypertrophy, parenchymal lesions, pleural angle bluntness, degenerative joint disease, emphysema changes, tracheal intubation, fractures, inflammatory reactions, malignant tumors, nasogastric tubes, osteoarthritis, osteoporosis, osteophytes, cardiac rhythm apparatus, catheter placement, drainage tubes, pleural effusions, pneumonia, pneumothorax, and artificial vascular implants, increased pulmonary striae, pulmonary edema, diaphragm hernia, vertebroarthropathy, sternotomy, tracheostomy, vertebroplasty, septum enlargement, or a combination thereof.

4. The auxiliary assess method according to claim 1, wherein each of the chest X-ray images is further annotated with a structural report of echocardiogram of a target disease.

5. The auxiliary assess method according to claim 4, wherein the target disease is selected from the group consisting of left ventricular dysfunction, aortic stenosis, pulmonary hypertension, left atrial enlargement, pericardial effusion, or a combination of the above.

6. The auxiliary assess method according to claim 1, wherein the step of using the contrastive learning model of the primary processing device to extract the high-level features from the preprocessed chest X-ray images comprises the step of using a small amount of case samples and another smaller amount of control samples wherein the ratio of the amount of the case samples to the amount of the control samples is 1:3.

7. The auxiliary assess method according to claim 6, wherein 128 case samples and 384 control samples are used, wherein secondary samples are randomly selected from the case and control samples by the ratio, starting from 1, wherein the secondary samples are oversampled.

8. An auxiliary assess system for conducting the auxiliary assess method according to claim 1 comprising:
an image acquisition device (10) for acquiring the chest X-ray images;
a data storage device (20) for storing the plural chest X-rays;
an image processing device (30) for preprocessing the chest X-ray images, thereby turning the chest X-ray images into preprocessed chest X-ray images in a same specification;
a primary processing device (40) for building the contrastive learning model and using the contrastive learning model to build the target prediction model;
a calculation device (50) for conducting the linear detection on the extracted high-level features of the processed chest X-ray images; and
an assess output device (60) for providing the assessed chest X-ray images.

9. The auxiliary assess system according to claim 8, wherein the image acquisition device, the data storage device, the image processing device, the processing device, the computing device and the assess output device are made in one piece.

10. The auxiliary assess method according to claim 8, wherein the image acquisition device, the data storage device, the image processing device, the processing device, the computing device and the assess output device are individual devise in communication of data with one another.
